(19) United States
(12) Patent Application Publication
(10) Pub. No.: US 2026/0363652 A1
(43) Pub. Date: Nov. 19, 2026

(54) BREAST TISSUE CHARACTERIZATION METHOD, DEVICE, MEDIUM, AND PRODUCT

(71) Applicant: Guangzhou Gesily Medical Technology Ltd., Guangzhou City (CN)

(72) Inventors: Xiaochuan LI, Guangzhou City (CN); Zhenyu GUO, Guangzhou City (CN); Ningtao ZHANG, Guangzhou City (CN); Jingli LIU, Guangzhou City (CN)

(21) Appl. No.: 19/446,088

(22) Filed: Jan. 12, 2026

(30) Foreign Application Priority Data
May 16, 2025 (CN) 202510634707.6

(51) Int. Cl. A61B 8/08; A61B 8/00; A61B 8/06; A61B 8/14; G06T 7/00; G06T 7/12; G06V 10/25; G06V 10/774; G06V 10/776; G06V 10/82

(52) U.S. Cl. CPC A61B 8/0825; A61B 8/06; A61B 8/145; A61B 8/483; A61B 8/485; A61B 8/488; A61B 8/5207; A61B 8/5223; A61B 8/5253; A61B 8/58; G06T 7/0012; G06T 7/12; G06V 10/25; G06V 10/774; G06V 10/776; G06V 10/82; G06T 2207/10136; G06T 2207/20081; G06T 2207/20084; G06T 2207/30068; G06T 2207/30096; G06V 2201/03

(57) ABSTRACT

Provided are a breast tissue characterization method, a device, a medium, and a product, relating to the field of breast tissue analysis. The method includes: determining RF signals, envelope signals, and B-mode signals corresponding to a cross section, a sagittal section, and a coronal section of a breast based on three-dimensional data of the breast; generating a corresponding B-mode image based on the determined B-mode signal of each section; inputting the B-mode images of all the sections to a region of interest (ROI) detection model to obtain a tumor ROI area; extracting from the three-dimensional data of the breast RF signals, envelope signals, and B-mode signals respectively corresponding to cross section, sagittal section, and coronal section ROIs based on the tumor ROI area; and inputting the extracted signals to a tumor tissue analysis model to obtain a tumor tissue characterization result.

### CROSS REFERENCE TO RELATED APPLICATION

This patent application claims the benefit and priority of Chinese Patent Application No. 202510634707.6, filed with the China National Intellectual Property Administration on May 16, 2025, the disclosure of which is incorporated by reference herein in its entirety as part of the present application.

### TECHNICAL FIELD

The present disclosure relates to the field of breast tissue analysis, and in particular, to abreast tissue characterization method, a device, a medium, and a product.

### BACKGROUND

The progression of breast cancer is a multi-stage and multi-step process. Most breast cancer patients initially develop abnormal hyperplasia of cells in the glands and lactiferous ducts, leading to atypical hyperplasia. This process belongs to the early stage of breast cancer formation. If the tumor is not detected promptly in a patient, it may progress into carcinoma in situ. Without timely treatment, the cancer cells will protrude from the ductal walls and even metastasize via blood and lymph, eventually evolving into advanced breast cancer. Therefore, early localization and tissue characterization of breast tumors can be conducive to clinical diagnosis, improving both survival rates and quality of life for breast cancer patients.

Currently, non-invasive methods for breast tumor localization and tissue characterization mainly include mammography, breast magnetic resonance imaging (MRI), and breast ultrasound imaging. However, mammography involves radiation risks and has low sensitivity to dense glands. Breast MRI is costly, requires expensive equipment, involves lengthy and expensive training for radiologists, and has certain patient limitations (e.g., contraindications for patients with metal implants, claustrophobia, or contrast agent allergies). Ultrasound imaging offers advantages such as no ionizing radiation, low cost, ease of operation, and accurate lesion localization, making it widely used in automated localization and tissue characterization in breast tumor research.

Nevertheless, conventional ultrasound imaging faces two major challenges in breast tumor localization and tissue characterization. First, clinicians must repeatedly examine images to determine the precise tumor location, and variations in experience and visual fatigue can lead to missed or misdiagnosed cases. Second, after acquiring breast imaging data, the analysis of tissue characteristics of breast tumors relies entirely on the subjective judgment of clinicians, lacking objective quantitative analysis of tumors. Thus, how to improve the efficiency and accuracy of breast tumor detection and localization, as well as enable quantitative analysis of tumor tissue characteristics, remains a challenging and unresolved issue.

### SUMMARY

An objective of the present disclosure is to provide abreast tissue characterization method, a device, a medium, and a product so as to improve the efficiency and accuracy of breast tumor detection and localization and enable quantitative analysis of tumor tissue characteristics.

To achieve the above objective, the present disclosure provides the following solutions.

A breast tissue characterization method includes the following steps:
acquiring three-dimensional data of a breast;
determining signals of sections of the breast based on the three-dimensional data of the breast, where the sections include a cross section, a sagittal section, and a coronal section, and the signals include a radio frequency (RF) signal, an envelope signal, and a B-mode signal;
generating a corresponding B-mode image based on the determined B-mode signal of each section;
inputting the B-mode images of all the sections to a region of interest (ROI) detection model to obtain a tumor ROI area, where the ROI detection model includes a tumor B-mode edge detection model configured to input the B-mode images of all the sections and output a tumor B-mode edge, a tracking module configured to obtain a tracked tumor envelope edge by tracking based on the tumor B-mode edge and obtain a tracked tumor RF edge by tracking based on the tracked tumor envelope edge, and an ROI generation module configured to determine the tumor ROI area based on the tumor B-mode edge, the tracked tumor envelope edge, and the tracked tumor RF edge;
extracting target signals from the three-dimensional data of the breast, where the target signals include RF signals, envelope signals, and B-mode signals respectively corresponding to a cross section ROI, a sagittal section ROI, and a coronal section ROI that are determined based on the tumor ROI area; the cross section ROI includes a region of a tumor in the cross section, the sagittal section ROI includes a region of the tumor in the sagittal section, and the coronal section ROI includes a region of the tumor in the coronal section; and
inputting the target signals to a tumor tissue analysis model and outputting a final tumor tissue characterization result by the tumor tissue analysis model, where the final tumor tissue characterization result includes a tumor morphology, a tumor aspect ratio, a tumor edge, tumor internal echo and tumor posterior echo characteristics, a calcification characteristic for characterizing whether the tumor is internally calcified, an invasiveness characteristic for characterizing whether the tumor is invasive, and a metastasis characteristic for characterizing whether the tumor metastasizes;
where the tumor tissue analysis model includes a quantitative characterization module configured to extract specific features of the tumor based on the target signals, a trained neural network configured to obtain a preliminary tumor tissue characterization result based on the target signals, and a trained systematic learning network configured to output the final tumor tissue characterization result based on the specific features and the preliminary tumor tissue characterization result.

Optionally, the ROI detection model is determined according to following steps: acquiring first training data and a first label, where the first training data includes the B-mode images of the sections of the breast determined based on the three-dimensional data of the breast as a sample, and the first label includes a true tumor B-mode edge;
constructing a target model, where the target model includes a first neural network for tumor edge detection, the tracking module, a tracing module, and the ROI generation module; an output of the first neural network for tumor edge detection is connected to an input to the tracking module; outputs of the first neural network for tumor edge detection, the tracking module, and the tracing module are all connected to the ROI generation module; the tracing module is configured to obtain a corresponding traced tumor envelope edge by tracing based on an input predicted tumor RF edge and obtain a traced tumor B-mode edge by tracing based on the obtained traced tumor envelope edge, and the predicted tumor RF edge is determined by a tumor RF edge detection model based on RF images corresponding to the B-mode images in the training data;
training the first neural network for tumor edge detection with the first training data and the first label for a plurality of times with an objective of minimizing a total loss to obtain the tumor B-mode edge detection model;
where a total loss in any training process is determined based on an error value which includes: an error between the tumor B-mode edge output by the first neural network for tumor edge detection obtained by last training and the true tumor B-mode edge, an error between the tracked tumor envelope edge generated by the tracking module and the predicted tumor envelope edge, an error between the tracked tumor RF edge generated by the tracing module and the predicted tumor RF edge, an error between the traced tumor envelope edge generated by the tracing module and the predicted tumor envelope edge, and an error between the traced tumor B-mode edge generated by the tracing module and the true tumor B-mode edge; and
connecting an output of the tumor B-mode edge detection model to the input to the tracking module, and connecting the outputs of the tumor B-mode edge detection model and the tracking module to the ROI generation module to obtain the ROI detection model.

Optionally, the error value further includes an error between the tumor B-mode edge output by the first neural network for tumor edge detection obtained by last training and an artificial feedback tumor edge determined based on artificial feedback information for the tumor ROI area.

Optionally, the quantitative characterization module includes first to third quantitative characterization modules, where the first quantitative characterization module is configured to extract a first specific feature of the tumor based on the RF signals of the target signals;
the second quantitative characterization module is configured to extract a second specific feature of the tumor based on the envelope signals of the target signals; and
the third quantitative characterization module is configured to extract a third specific feature of the tumor based on the B-mode signals of the target signals.

Optionally, the trained neural network includes first to third trained neural networks, where the first trained neural network is configured to obtain, based on the RF signals of the target signals, a preliminary tumor tissue characterization result corresponding to the RF signals;
the second trained neural network is configured to obtain, based on the envelope signals of the target signals, a preliminary tumor tissue characterization result corresponding to the envelope signals; and
the third trained neural network is configured to obtain, based on the B-mode signals of the target signals, a preliminary tumor tissue characterization result corresponding to the B-mode signals.

Optionally, the trained systematic learning network specifically includes a feature selection module, a trained first-layer primary learner, and a trained second-layer meta learner that are connected in sequence;
the feature selection module is configured to select, using a mutual information method, the input specific features and preliminary tumor tissue characterization results to obtain a selection result;
the trained first-layer primary learner is configured to input the selection result and output a primary tumor tissue characterization result; and
the trained second-layer meta learner is configured to input the primary tumor tissue characterization result and output the final tumor tissue characterization result.

Optionally, the determining signals of sections of the breast specifically includes: extracting the RF signal of each section of the breast from the three-dimensional data of the breast;
performing pre-gain amplification on the RF signal of each section, demodulating the amplified signal using an in-phase and quadrature demodulation algorithm, and detecting the demodulated signal by hilbert transform to obtain the envelope signal of each section of the breast; and
performing logarithmic compression on the envelope signal of each section to obtain the B-mode signal of each section of the breast.

Optionally, the acquiring three-dimensional data of a breast specifically includes: scanning the breast using a multimodal breast volumetric ultrasound integrated machine to obtain the three-dimensional data of the breast.

Optionally, the extracting target signals from the three-dimensional data of the breast specifically includes: receiving artificial calibration information for the tumor ROI area;
determining a final tumor ROI area based on the artificial calibration information, where the final tumor ROI area includes the cross section ROI, the sagittal section ROI, and the coronal section ROI; and
extracting from the three-dimensional data of the breast the RF signal, the envelope signal, and the B-mode signal corresponding to the cross section ROI, the RF signal, the envelope signal, and the B-mode signal corresponding to the sagittal section ROI, and the RF signal, the envelope signal, and the B-mode signal corresponding to the coronal section ROI.

Optionally, the first specific feature includes a complexity related feature, a texture related feature, a frequency domain related feature, and a blood flow doppler related feature;
the second specific feature includes a shape parameter and a scale parameter in a distribution model; and
the third specific feature includes an elasticity imaging related parameter, a texture related feature, a morphology related feature, and an image gray level related feature.

The present disclosure further provides a computer device, including a memory, a processor, and a computer program stored on the memory and runnable on the processor, where the processor is configured to execute the computer program to implement the breast tissue characterization method described above.

The present disclosure further provides a computer-readable storage medium, storing thereon a computer program which, when executed by a processor, implements the breast tissue characterization method described above.

The present disclosure further provides a computer program product, including a computer program which, when executed by a processor, implements the breast tissue characterization method described above.

According to specific embodiments provided by the present disclosure, the present disclosure has the following technical effects: in the embodiments of the present disclosure, the B-mode images of the breast in the cross section, the sagittal section, and the coronal section are extracted based on the three-dimensional data of the breast, and the tumor ROI area is predicted by the ROI detection model based on the B-mode images. Compared with clinician's judgment with eyes, the efficiency and accuracy of breast tumor detection and localization are improved. Furthermore, the specific features are extracted using the quantitative characterization modules in the tumor tissue analysis model, allowing for signal quantization characterization of the tumor ROI areas corresponding to the sections. Besides, quantitative analysis of the tumor tissue characteristics is enabled by using the trained neural networks in combination with the trained systematic learning network.

### BRIEF DESCRIPTION OF THE DRAWINGS

FIG. 1 is a flowchart of a breast tissue characterization method provided by Embodiment 1 of the present disclosure;

FIG. 2 is a schematic diagram of construction of an ROI detection model;

FIG. 3 is a schematic diagram of a training process of the ROI detection model;

FIG. 4 is a schematic diagram of tracing learning;

FIG. 5 is a schematic diagram of an ROI;

FIG. 6 is a schematic diagram of signals extracted from an ROI;

FIG. 7 is a schematic diagram of construction of a tumor tissue analysis model;

FIG. 8 is a schematic diagram of systematic learning;

FIG. 9 is a flowchart of specific application of a tissue characterization method; and

FIG. 10 is a diagram of an internal structure of a computer device.

### DETAILED DESCRIPTION OF THE EMBODIMENTS

The technical solutions in the embodiments of the present disclosure will be described below in combination with the accompanying drawings in the embodiments of the present disclosure.

An objective of the present disclosure is to provide abreast tissue characterization method, a device, a medium, and a product, aiming to improve the efficiency and accuracy of breast tumor detection and localization and enable quantitative analysis of tumor tissue characteristics.

To make the above objectives, features, and advantages of the present disclosure clearer and more comprehensible, the present disclosure will be further described in detail below with reference to the accompanying drawings and the specific embodiments.

First, the overall idea of a breast tissue characterization method provided in the present embodiment is illustrated.

The breast tissue characterization method in the present embodiment can enable retrospective feedback learning with different sections of three-dimensional data of a breast, achieve automatic partitioning of tumor regions of interest, and enable systematic learning with the tumor regions of interest to realize breast tumor tissue characterization. This method mainly involves two models: a Region of Interest (ROI) detection model and a tumor tissue analysis model.

The ROI detection model is obtained by first acquiring three-dimensional data of a breast by using a multimodal breast volumetric ultrasound integrated machine, then extracting a cross section, a sagittal section, and a coronal section from the acquired three-dimensional data of the breast to obtain a radio frequency (RF) image, an envelope image, and a B-mode image of each section, performing supervised retrospective learning based on artificial calibration with all the images as inputs, and outputting corresponding tumor B-mode edge, tumor envelope edge, and tumor RF edge.

The tumor tissue analysis model is obtained according to the following steps: the RF signals, the envelope signals, and the B-mode signals corresponding to 3 sections of the ROI obtained based on the ROI detection model are extracted first. Each extracted signal is subjected to quantitative characterization to obtain different specific features. The specific features are then used as inputs to a neural network and subjected to supervised retrospective learning based on artificial calibration, thereby obtaining the tumor tissue analysis model.

In practical application, the resulting three-dimensional data of the breast to be characterized is input to the above trained ROI detection model and tumor tissue analysis model to obtain a final tumor tissue characterization result.

This method accurately enables tissue characterization of a breast tumor, quantitative detection of a tumor location, and analysis of the tumor tissue. The method is simple, and convenient to operate, and can achieve automatic scanning. By this method, the breast tumor ROI and the edge details are highlighted. The changes of acoustic features of the tumor tissue structure are analyzed accurately. A new method and theoretical basis are provided for clinically reducing missed diagnosis and misdiagnosis rates.

Several embodiments are provided to illustrate the present disclosure below for the purpose of practical application.

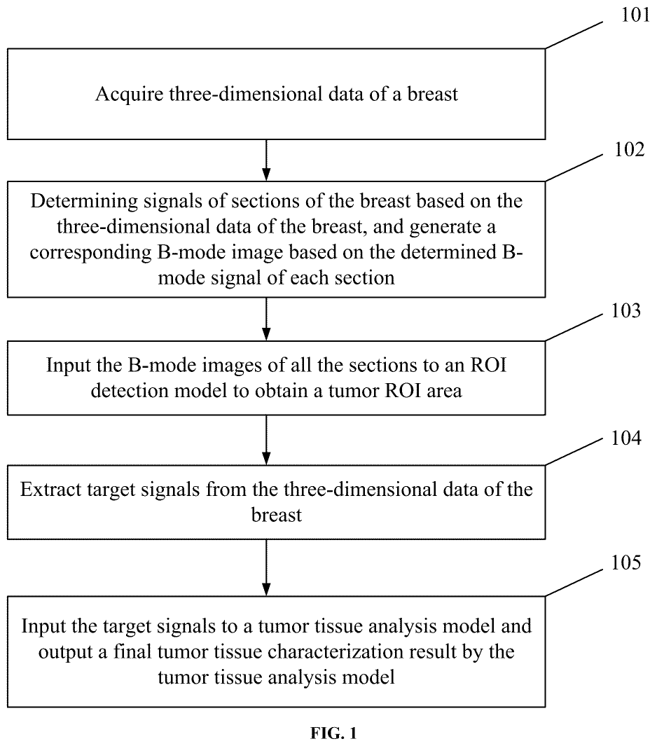

Embodiment 1: as shown in FIG. 1, a breast tissue characterization method provided in the present embodiment includes the following steps.

In step 101, three-dimensional data of a breast is acquired.

In one example, the breast tumor is automatically scanned using the multimodal breast volumetric ultrasound integrated machine to obtain the three-dimensional data of the breast. In the scanning process, there is no need for the professional clinician to search for the tumor. Rather, the three-dimensional data of the breast can be obtained by placing a probe on the breast for about 30 s.

In another example, in order to increase the frame frequency and improve the imaging quality, when scanning by the multimodal breast volumetric ultrasound integrated machine, in addition to focused ultrasound, plane wave ultrasound is used in combination for scanning. That is, scanning is implemented using breast 3D volume focusing/plane wave ultrasound. In particular, the breast is first scanned once using focused ultrasound. Then, a multi-angle plane wave coherent composite imaging method is adopted to emit a plurality of plane waves with different deflection angles towards the breast. Echo signals from the emitted plane waves are combined into a wave beam, followed by coherent combination. By artificial judgment, the data with the best effect of the three-dimensional data of the breast output from the above two scans is used as the final three-dimensional data of the breast. Subsequently, RF images may be obtained by performing a series of signal processing operations on the three-dimensional data of the breast (i.e., breast RF signals). For example, the signal processing operations may include: B-mode envelope detection, Color-mode autocorrelation processing, and processing by a digital signal controller (DSC), etc.

In step 102, signals of sections of the breast are determined based on the three-dimensional data of the breast, and a corresponding B-mode image is generated based on the determined B-mode signal of each section. The sections include a cross section, a sagittal section, and a coronal section, and the signals include an RF signal, an envelope signal, and a B-mode signal.

In one example, the determination of the signals of the sections of the breast specifically includes the following steps: (1) the RF signal of each section of the breast is extracted from the three-dimensional data of the breast.

(2) Pre-gain amplification is performed on the RF signal of each section. The amplified signal is demodulated using an in-phase and quadrature demodulation algorithm. The demodulated signal is detected by hilbert transform to obtain the envelope signal of each section of the breast. A calculation formula of the hilbert transform is as follows:

$$H ( u ) ( t ) = u * h = \frac{1}{π} P {\int }_{- ∞}^{+ ∞} \frac{u ( τ )}{t - τ} d τ , \qquad ( 1 )$$

where u represents the demodulated signal; h represents a signal for convolution; regarding

$$h = \frac{1}{π t} ,$$

t represents time, and τ represents an integration variable; P represents a Cauchy's principal value; and H(u)(t) represents an analytic signal after the hilbert transform, i.e., the envelope signal.

(3) Logarithmic compression is performed on the envelope signal of each section to obtain the B-mode signal of each section of the breast. A calculation formula of the logarithmic compression is as follows:

Y(t)=20*logₑ(H(u)(t)) (2),
where Y(t) represents the B-mode signal.

As can be seen, in this example, the cross section, the sagittal section, and the coronal section are extracted from the obtained three-dimensional data of the breast, and one RF signal, one envelope signal, and one B-mode signal are obtained based on each section. That is, corresponding 3 RF signals, 3 envelope signals, and 3 B-mode signals are obtained for the 3 sections.

Subsequently, a corresponding RF image, envelope image, and B-mode image may be generated based on the RF signal, the envelope signal, and the B-mode signal of each section. That is, corresponding 3 RF images, 3 envelope images, and 3 B-mode images are obtained for the 3 sections.

Relations of the RF image, the envelope image, and the B-mode image are as follows: (1) the RF image is an image generated from the RF signal without any processing. (2) The envelope image is an image generated from the envelope signal. (3) The B-mode image is an image generated from the B-mode signal.

In step 103, the B-mode images of all the sections are input to an ROI detection model to obtain a tumor ROI area.

The ROI detection model includes a tumor B-mode edge detection model configured to input the B-mode images of all the sections and output a tumor B-mode edge, a tracking module configured to obtain a tracked tumor envelope edge by tracking based on the tumor B-mode edge and obtain a tracked tumor RF edge by tracking based on the tracked tumor envelope edge, and an ROI generation module configured to determine the tumor ROI area based on the tumor B-mode edge, the tracked tumor envelope edge, and the tracked tumor RF edge.

In one example, the ROI detection model is determined by a process specifically including the following steps.

(1) First training data and a first label are acquired, where the first training data includes the B-mode images of the sections of the breast determined based on the three-dimensional data of the breast as a sample, and the first label includes a true tumor B-mode edge.

(2) A target model is constructed. The target model includes a first neural network for tumor edge detection, the tracking module, a tracing module, and the ROI generation module. An output of the first neural network for tumor edge detection is connected to an input to the tracking module. Outputs of the first neural network for tumor edge detection, the tracking module, and the tracing module are all connected to the ROI generation module. The tracing module is configured to obtain a corresponding traced tumor envelope edge by tracing based on an input predicted tumor RF edge and obtain a traced tumor B-mode edge by tracing based on the obtained traced tumor envelope edge, and the predicted tumor RF edge is determined by a tumor RF edge detection model based on RF images corresponding to the B-mode images in the training data. The first neural network for tumor edge detection may include a convolutional layer, a pooling layer, and a fully connected layer.

(3) The first neural network for tumor edge detection is trained with the first training data and the first label for a plurality of times with an objective of minimizing a total loss to obtain the tumor B-mode edge detection model.

A total loss in any training process is determined based on an error value which includes: an error between the tumor B-mode edge output by the first neural network for tumor edge detection obtained by last training and the true tumor B-mode edge, an error between the tracked tumor envelope edge generated by the tracking module and the predicted tumor envelope edge, an error between the tracked tumor RF edge generated by the tracing module and the predicted tumor RF edge, an error between the traced tumor envelope edge generated by the tracing module and the predicted tumor envelope edge, and an error between the traced tumor B-mode edge generated by the tracing module and the true tumor B-mode edge.

An output of the tumor B-mode edge detection model is connected to the input to the tracking module, and the outputs of the tumor B-mode edge detection model and the tracking module are connected to the ROI generation module to obtain the ROI detection model.

The tumor RF edge detection model used above is determined by a process specifically including the following steps.

Second training data and a second label are acquired, where the second training data includes the RF images of the sections of the breast determined based on the three-dimensional data of the breast as the sample, and the second label includes a true tumor RF edge.

A second neural network for tumor edge detection is trained with the second training data and the second label for a plurality of times with an objective of minimizing an RF edge loss to obtain the tumor RF edge detection model. The second neural network for tumor edge detection may have the same network structure with the first neural network for tumor edge detection.

The RF edge loss is determined based on an error between the predicted tumor RF edge output by the neural network for tumor edge detection and the true tumor RF edge.

The tumor envelope edge detection model used above is determined by a process specifically including the following steps.

Third training data and a third label are acquired, where the third training data includes the envelope images of the sections of the breast determined based on the three-dimensional data of the breast as the sample, and the third label includes a true tumor envelope edge.

A third neural network for tumor edge detection is trained with the third training data and the third label for a plurality of times with an objective of minimizing an envelope edge loss to obtain the tumor envelope edge detection model. The third neural network for tumor edge detection may have the same network structure with the first neural network for tumor edge detection.

The envelope edge loss is determined based on an error between the predicted tumor envelope edge output by the neural network for tumor edge detection and the true tumor envelope edge.

In another example, the error value further includes an error between the tumor B-mode edge output by the first neural network for tumor edge detection obtained by last training and an artificial feedback tumor edge determined based on artificial feedback information for the tumor ROI area.

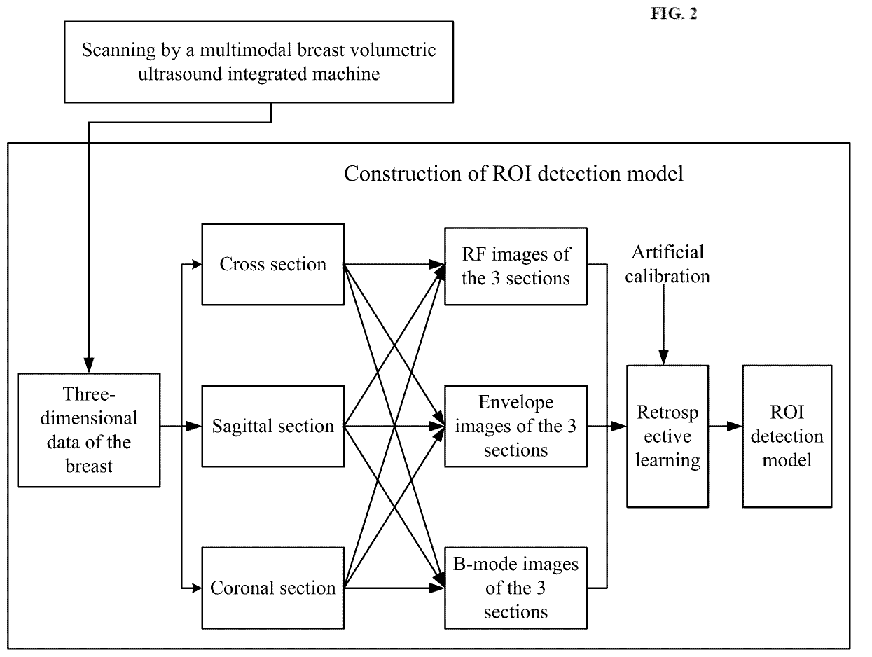

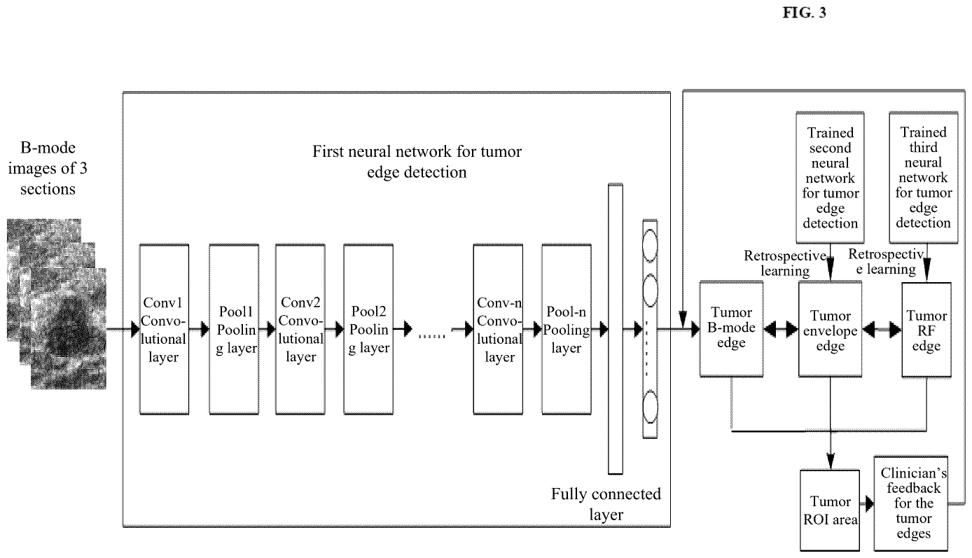

The training process of the ROI detection model is further illustrated below in detail with reference to FIG. 2 and FIG. 3.

The B-mode images of the 3 sections, the envelope images of the 3 sections, and the RF images of the 3 sections are used as inputs for supervised retrospective learning in combination with artificial calibration, as shown in FIG. 2 and FIG. 3. The B-mode images of the 3 sections are passed through the convolutional layer, the pooling layer, and the fully connected layer of the first neural network for tumor edge detection to obtain the tumor B-mode edge. Based on the tumor B-mode edge, the predicted tumor envelope edge output by the trained second neural network for tumor edge detection, and the predicted tumor RF edge output by the trained third neural network for tumor edge detection, the tumor envelope edge and the tumor RF edge are obtained through retrospective learning. Subsequently, the tumor ROI area is determined through the ROI generation module.

The first neural network for tumor edge detection, the second neural network for tumor edge detection, and the third neural network for tumor edge detection have the same network structure. Taking the first neural network for tumor edge detection as an example, it includes n convolutional layers (Conv1 convolutional layer, Conv2 convolutional layer, . . . , and Convn convolutional layer), n pooling layers (Pool1 pooling layer, Pool2 pooling layer, . . . , and Pool-n pooling layer), and one fully connected layer. The connection relationship of these layer structures is as shown in FIG. 3.

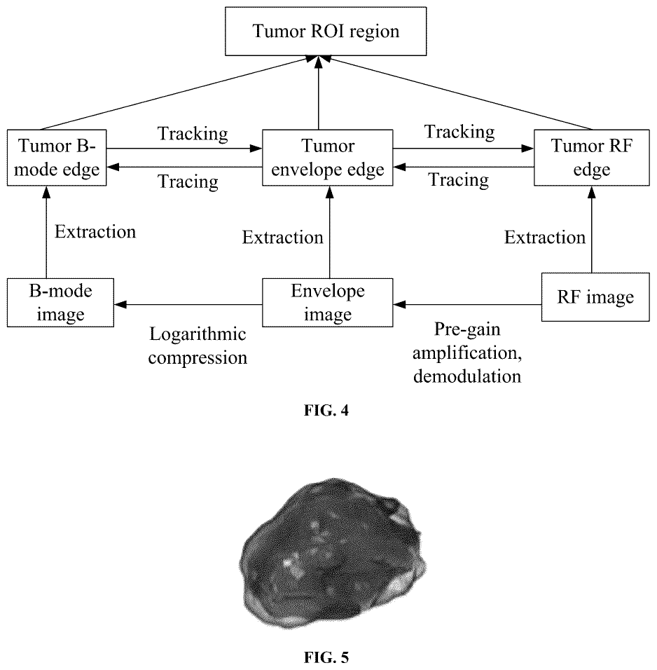

The retrospective learning includes two parts: tracking and tracing. The tracking refers to forward propagation of information in a signal from up to down. By following a specific rule or source of information, for example, in accordance with the tumor B-mode edge, the tracked tumor envelope edge is obtained correspondingly to the tumor envelope edge, and with the tracked tumor envelope edge, the tracked tumor RF edge is obtained correspondingly to the tumor RF edge. The tracing refers to backpropagation of the information in the signal from down to up, i.e., cyclic feedback capability for the information. For example, with the tumor RF edge, the traced tumor envelope edge is obtained correspondingly to the tumor envelope edge, and with the traced tumor envelope edge, the traced tumor B-mode edge is obtained correspondingly to the tumor B-mode edge, as shown in FIG. 4. The process of continuous iteration in the retrospective learning may be expressed by formula (3):

Q₂(s,a)←Q₁(s,a)+α[R+γ max_{a′}Q(s′,a′)−Q(s,a)] (3),
where Q₂(s, a) represents a new value of signal a in state s; Q₁(s, a) represents an original value of the signal a in the state s; R is a reward obtained based on a; max_{a′}Q(s′, a′) represents a maximum value of all signals in next state s′; and α and γ are both parameters, 0≤α≤1, and 0≤γ≤1.

Taking FIG. 4 as an example, the formula (3) represents iteration processes in two tracing processes from the tumor RF edge to the tumor envelope edge and from the tumor envelope edge to the tumor B-mode edge. When tracing from the tumor RF edge to the tumor envelope edge, Q₂(s, a) represents an accurate tumor envelope edge, i.e., a label of the tumor envelope edge, Q₁ (s, a) represents an unadjusted tumor RF edge, Q(s′, a′) represents the tumor envelope edge obtained by adjusting Q₁(s, a) once, and Q(s, a) represents the tumor envelope edge obtained after the last adjustment of Q(s′, a′). The formula for tracing from the tumor envelope edge to the tumor B-mode edge is similar to the formula (3), which will not be described here redundantly.

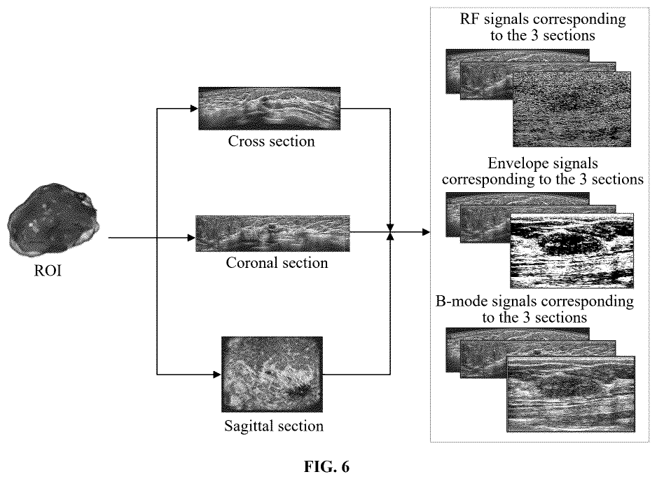

Next, the tumor is localized based on the tumor edges obtained from the 9 images corresponding to the 3 sections. Finally, the resulting tumor area and the surrounding invaded tissue is regarded as the ROI. Thus, the ROI detection model is obtained. The ROI is as shown in FIG. 5. Artificial calibration is conducted by the clinician based on the B-mode image, which is regarded as a label in the retrospective learning process for providing feedback of the obtained tumor location. Thus, the accuracy of the edge detection result is improved. A transfer function that depicts a relationship between an input and an output in a feedback algorithm is expressed as formula (4):

G(s)=H(s)/(1+H(s)*F(s)) (4).

Corresponding to FIG. 3, in the formula (4), G(s) denotes a transfer function for the feedback part, representing the ROI detection model, H(s) denotes a transfer function for a feedback path, representing a clinician's feedback for the tumor edge, and F(s) denotes a transfer function for a forward path, representing the tumor ROI area obtained through the retrospective learning.

In step 104, target signals are extracted from the three-dimensional data of the breast.

The target signals include RF signals, envelope signals, and B-mode signals respectively corresponding to a cross section ROI, a sagittal section ROI, and a coronal section ROI that are determined based on the tumor ROI area. The cross section ROI includes a region of a tumor in the cross section, the sagittal section ROI includes a region of the tumor in the sagittal section, and the coronal section ROI includes a region of the tumor in the coronal section.

In one example, the above step 104 specifically includes the following steps.

Artificial calibration information for the tumor ROI area is received.

A final tumor ROI area is determined based on the artificial calibration information. The final tumor ROI area includes the cross section ROI, the sagittal section ROI, and the coronal section ROI.

The RF signal, the envelope signal, and the B-mode signal corresponding to the cross section ROI, the RF signal, the envelope signal, and the B-mode signal corresponding to the sagittal section ROI, and the RF signal, the envelope signal, and the B-mode signal corresponding to the coronal section ROI are extracted from the three-dimensional data of the breast.

In particular, after the ROI model is obtained, the RF signals, envelope signals, and B-mode signals respectively corresponding to the cross section, the sagittal section, and the coronal section, a total of 9 signals, are extracted based on the ROIs. FIG. 6 is a schematic diagram of extraction of one tumor ROI. First, RF data of the cross section, the sagittal section, and the coronal section of a three-dimensional ROI is obtained by a projection method. Pre-gain amplification and demodulation are then performed based on the RF data of the 3 sections to obtain the envelope signals of the 3 sections, followed by logarithmic compression of the envelope signals to obtain the B-mode signals of the 3 sections. That is, a total of 9 signals are extracted, and the 9 signals are separately subjected to quantitative characterization and learning via a neural network.

In step 105, the target signals are input to a tumor tissue analysis model and a final tumor tissue characterization result is output by the tumor tissue analysis model.

The final tumor tissue characterization result includes a tumor morphology, a tumor aspect ratio, a tumor edge, tumor internal echo and tumor posterior echo characteristics, a calcification characteristic for characterizing whether the tumor is internally calcified, an invasiveness characteristic for characterizing whether the tumor is invasive, and a metastasis characteristic for characterizing whether the tumor metastasizes.

The tumor tissue analysis model includes a quantitative characterization module configured to extract specific features of the tumor based on the target signals, a trained neural network configured to obtain a preliminary tumor tissue characterization result based on the target signals, and a trained systematic learning network configured to output the final tumor tissue characterization result based on the specific features and the preliminary tumor tissue characterization result.

In one example, the quantitative characterization module includes first to third quantitative characterization modules.

The first quantitative characterization module is configured to extract a first specific feature of the tumor based on the RF signals of the target signals. The first specific feature may include, for example, a complexity related feature, a texture related feature, a frequency domain related feature, and a blood flow doppler related feature.

The second quantitative characterization module is configured to extract a second specific feature of the tumor based on the envelope signals of the target signals. The second specific feature may include, for example, a shape parameter and a scale parameter in a distribution model.

The third quantitative characterization module is configured to extract a third specific feature of the tumor based on the B-mode signals of the target signals. The third specific feature may include, for example, an elasticity imaging related parameter, a texture related feature, a morphology related feature, and an image gray level related feature.

The trained neural network includes first to third trained neural networks.

The first trained neural network is configured to obtain, based on the RF signals of the target signals, a preliminary tumor tissue characterization result corresponding to the RF signals. The second trained neural network is configured to obtain, based on the envelope signals of the target signals, a preliminary tumor tissue characterization result corresponding to the envelope signals. The third trained neural network is configured to obtain, based on the B-mode signals of the target signals, a preliminary tumor tissue characterization result corresponding to the B-mode signals.

The trained systematic learning network specifically includes a feature selection module, a trained first-layer primary learner, and a trained second-layer meta learner that are connected in sequence. The degree of fitting of the first-layer primary learner is greater than that of the second-layer meta learner. The second-layer meta learner is a generalized linear model.

The feature selection module is configured to select, using a mutual information method, the input specific features and preliminary tumor tissue characterization results to obtain a selection result. The trained first-layer primary learner is configured to input the selection result and output a primary tumor tissue characterization result. The trained second-layer meta learner is configured to input the primary tumor tissue characterization result and output the final tumor tissue characterization result.

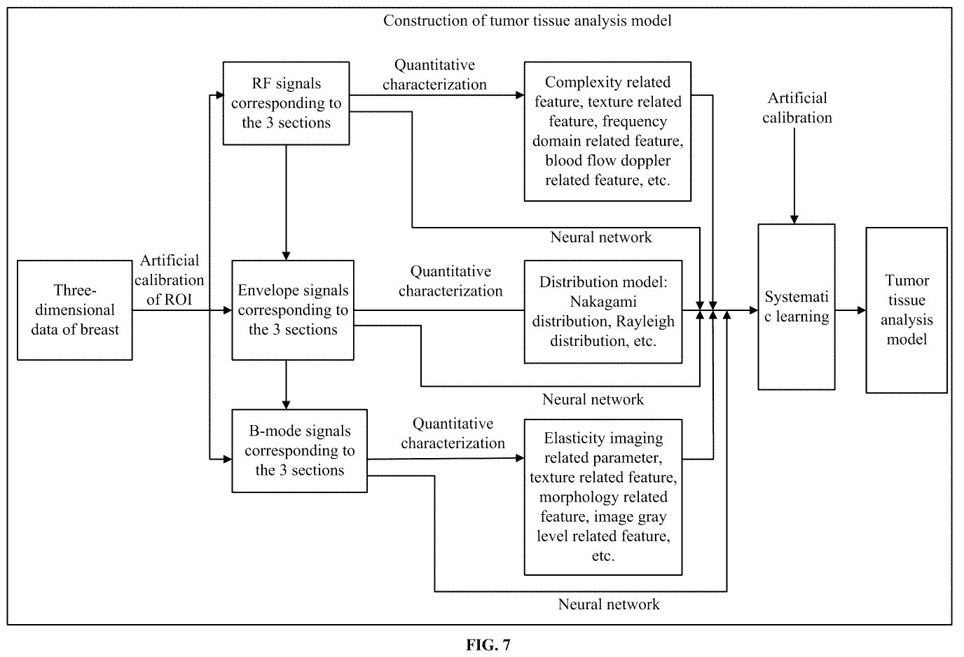

The training process of the tumor tissue analysis model is as shown in FIG. 7.

Next, the first specific feature and the first trained neural network mentioned above are described in detail.

(1) The complexity related feature includes an entropy, a Rayleigh entropy, a cross entropy, and a cumulative residual entropy. A calculation formula for the entropy H_{c} is as shown in formula (5), a calculation formula for the Rayleigh entropy R_{β}(Y) is as shown in formula (6), a calculation formula for the cross entropy H(p, q) is as shown in formula (7), and a calculation formula for the cumulative residual entropy ε(Y) is as shown in formula (8):

$${H}_{c} = - {\sum }_{y ∈ Y} w ( y ) {log}_{2} ( w ( y ) ) , \qquad ( 5 )$$

$${R}_{β} ( Y ) = \frac{1}{1 - β} log {\sum }_{y ∈ Y} {w ( y )}^{β} , \qquad ( 6 )$$

$$H ( p , q ) = - {\sum }_{y ∈ Y} p ( y ) {log}_{2} ( q ( y ) ) , \qquad ( 7 )$$

$$and$$

$$ε ( Y ) = - {\sum }_{y ∈ Y} W ( Y > y ) log Y ( Y > y ) , \qquad ( 8 )$$

where Y represents a number of intervals an RF signal amplitude range is divided into; w(y) represents a statistical histogram of an RF signal; β>0, β≠1; p represents a real probability distribution of a feature; q represents a non-real probability distribution of the feature; and W represents a cumulative probability distribution of the RF signal.

(2) The texture related feature includes energy, correlation, and homogeneity features obtained based on a gray level concurrence matrix, a roughness, a contrast, and a directionality extracted based on a Tamura texture feature, and a space-domain feature extracted based on the mathematical morphology.

(3) The frequency domain related feature includes an energy concentration ratio, a frequency peak value, a spectrum width, a frequency domain coherence, and a spacing between scattering points.

(4) The blood flow doppler related feature includes whether the blood flow is rich, and a blood flow rate. On the other hand, the first specific feature is input to the constructed neural network for supervised learning with the signals in the directions of the 3 sections and the labels (i.e., the real tumor tissue characterization result) as a training set. After the model has been trained, it serves as the first trained neural network into which the RF signals of the different sections are input.

Next, the second specific feature and the second trained neural network mentioned above are described in detail.

With regard to the envelope signals corresponding to the 3 sections, on the one hand, a shape parameter m and a scale parameter Ω in Nakagami distribution are extracted, as shown in formula (9), and an aggregation parameter MU and a derivation parameter k=ε/(σ\sqrt{mu}) in homodyned K-distribution are used as parameter features, as shown in formula (10). On the other hand, the second specific feature is input to the constructed neural network for supervised learning with the signals in the directions of the 3 sections and the labels (i.e., the real tumor tissue characterization result) as the training set. After the model has been trained, it serves as the second trained neural network into which the envelope signals of the different sections are input.

$$f ( E ) = \frac{2 {m}^{2} {R}^{2 m - 1}}{Γ ( m ) {( Ω )}^{m}} exp ( - \frac{m}{Ω} {m}^{2} ) U ( E ) , \qquad ( 9 )$$

$$and$$

$${P}_{k} ( A \vert ε , {σ}^{2} , mu ) = \frac{{A}^{\frac{n}{2}}}{{( ε )}^{\frac{n}{2}}} \int μ {J}_{\frac{n}{2} - 1} ( με ) {J}_{\frac{n}{2} - 1} ( μ A ) {( 1 + \frac{{μ}^{2} {σ}^{2}}{2} )}^{- mu} d μ , \qquad ( 10 )$$

where E represents an envelope of a backscattered signal; Γ(m) represents Euler's Gamma function; ε² and σ² represent a coherent component and an incoherent component of a signal; ƒ (E) represents a probability density function of R; U(E) represents a standard uniform distribution function; Pₖ(A|ε, σ², mu) represents n-dimensional HK distribution; m represents the shape parameter; J represents zeroth-order Bessel function of the first kind; mμ represents the aggregation parameter; and A represents an envelope amplitude of the backscattered signal.

Next, the third specific feature and the third trained neural network mentioned above are described in detail.

With regard to the B-mode signals corresponding to the 3 sections, on the one hand, the specific features such as the elasticity imaging related parameter, the texture related feature, the morphology related feature, and the image gray level related feature are extracted. Next, the specific features included in these main classes are enumerated in detail: (1) the elasticity imaging related parameter is mainly obtained by calculating a value of Young's modulus E, as shown in formula (11); (2) the feature included in the texture related feature is the same as the texture feature extracted from the RF signal; (3) the morphology related feature mainly includes a tumor size, a tumor shape, and a tumor texture; and (4) the image gray level related feature mainly includes a gray level mean, a gray level variance, gray level energy, and a gray level entropy. On the other hand, the third specific feature is input to the constructed neural network for supervised learning with the signals in the directions of the 3 sections and the labels (i.e., the real tumor tissue characterization result) as the training set. After the model has been trained, it serves as the third trained neural network into which the B-mode signals of the different sections are input.

E=χ/η=(F/S)/(ΔL/L0)=FL0/SΔL (11),
where χ represents a uniaxial stress; η represents a strain; S represents a cross sectional area; F represents a compressing force or stretching force; ΔL represents a change in length; and L0 represents an original length.

Next, the above-mentioned systematic learning network is described in detail.

With the quantitative characterization parts and the neural network learned parts of the 9 signals as inputs, the clinician performs artificial calibration on different tumor tissue characteristics (the tumor morphology, the tumor aspect ratio, the tumor edge, the tumor internal echo and tumor posterior echo characteristics, whether the tumor is internally calcified, whether the tumor is invasive, and whether the tumor metastasizes), which are used as labels. Systematic learning is carried out to obtain the final tumor tissue analysis model.

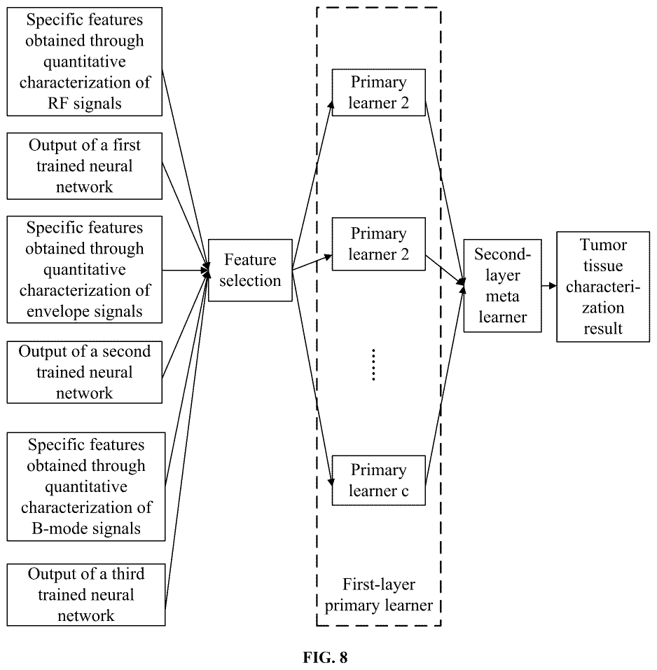

The systematic learning network is as shown in FIG. 8. First, feature selection is performed on all the specific features extracted from all the signals and the preliminary tumor tissue characterization results output by the neural networks using a mutual information method, as shown in formula (12). Next, using Stacking integrated learning method, the first-layer primary learner is constructed first, which includes c primary learners: primary learner 1, primary learner 2, . . . , and primary learner c. The second-layer meta learner is then constructed. A result of the first-layer primary learner is used as an input to the second-layer meta learner. In this way, the final systematic learning network is formed.

For the c primary learners in the first-layer primary learner, a model with a high degree of fitting is used, such as a neural network and a support vector machine, for fully learning training data and automatically extracting effective features. For the second-layer meta learner, a simple model is used, e.g., a generalized linear model such as logistic regression or Lasso regression. As a result, the risk of overfitting can be reduced. A tumor tissue analysis result is output, and a deterioration degree of the tumor is measured by determining whether the tumor is invasive and whether the tumor metastasizes.

$$I ( X , Z ) = {\sum }_{x ∈ X} {\sum }_{z ∈ Z} p ( x , z ) log \frac{p ( x , z )}{p ( x ) p ( z )} , \qquad ( 12 )$$

where X represents a feature set; Z represents a category set; I(X, Z) represents mutual information for feature section; p(x, z) represents a probability that the feature is x and the category is z; p(x) represents a probability that the feature is x; and p(z) represents a probability that the feature is z.

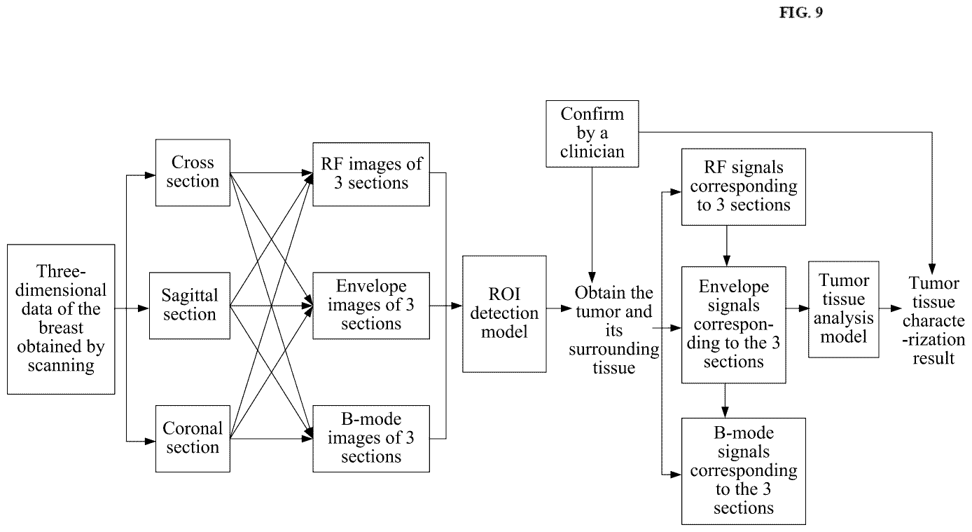

With reference to FIG. 9, the specific application process of the breast tissue characterization method is illustrated below.

In particular, first, focusing/plane wave ultrasonic scanning is performed on the breast using the multimodal breast volumetric ultrasound integrated machine to obtain the three-dimensional data of the breast. The RF images, the envelope images, and the B-mode images corresponding to the cross section, the sagittal section, and the coronal section of the breast extracted from the three-dimensional data of the breast are input to the trained ROI detection model to extract the tumor area and the surrounding tissue as the tumor ROI area. The extracted tumor ROI area is judged by the clinician.

Next, the tumor is subjected to tissue characterization. The corresponding RF signals, envelope signals, and B-mode signals in the directions of the 3 sections are extracted based on the tumor ROI area. These signals are input to the trained tumor tissue analysis model at the same time to obtain the tumor tissue characterization result of the breast. The tumor tissue characterization result is mainly used for describing a difference in acoustic characteristic between different tumors and analyzing the tissue structures of different tumors.

The training of the tissue characterization model and the practical application of the tissue characterization method are described in detail in the above embodiment.

The training of the tissue characterization model is further divided into the training of the ROI detection model and the training of the tumor tissue analysis model. The ROI detection model is obtained mainly by automatically extracting the tumor area and the surrounding tissue through retrospective learning. The tumor tissue analysis model is obtained mainly by extracting the RF signals, the envelope signals, and the B-mode signals in the directions of the 3 sections based on the tumor ROI area, subjecting all the signals to quantitative characterization and learning via the neural networks, and finally performing systematic learning with the specific features extracted from all the signals and the results after learning via the neural networks.

In the practical application of the tissue characterization method, principally, the acquired data is directly input to the trained ROI detection model to obtain the tumor ROI area. In this way, the dependence on the experience of the operating personnel is greatly reduced, the tumor detection and localization efficiency is improved, and the demand on sonographers is relieved. Based on the tumor ROI area, the extracted signals are input to the tumor tissue analysis model to obtain the final breast tumor tissue characterization result, whereby the breast tumor tissue is analyzed objectively and quantitatively. The tissue characterization model and method have very important research value and promising application prospects for clinical early screening, diagnosis, and treatment.

The advantages of the breast tissue characterization method provided in the above embodiment are described in detail below.

Currently, the use of conventional two-dimensional (2D) ultrasound for breast tumor localization and tissue characterization suffers from long scanning time and low efficiency. This process requires sonographers to invest significant time, and the quality of the resulting ultrasound imaging (i.e., the accuracy of the tumor location and the tissue characterization result) heavily depends on the subjective experience of the clinicians. Recently introduced breast volumetric ultrasound has partially addressed these issues. However, due to the loss of certain information during B-mode image generation and the fact that the 2D image quality (cross section and sagittal section) is not necessarily superior to that of the conventional 2D ultrasound, the delineation of the breast tumor location and the tumor tissue characterization still depend on the subjective experience of the clinicians. This approach lacks precise, quantitative, and objective descriptions. Although several methods for ROI extraction and tissue characterization based on quantitative ultrasound features have been proposed, the analysis of the tumor tissue using only one feature or several features still have certain restrictions.

In the present embodiment, focusing/plane wave ultrasonic scanning is performed on the breast using the multimodal breast volumetric ultrasound integrated machine. The tumor in the breast is localized and the edges are detected by means of the retrospective learning network, and the extracted edges are adjusted with the clinician's feedback, and different signals corresponding to the directions of different sections are subjected to quantitative characterization and learning via the neural networks. Systematic learning is then performed on all the signals corresponding to all the sections. By making full use of the advantages of each section and each signal, the problems of long scanning time, strong subjectivity, insufficient image quality, and incomplete information are effectively solved. Automatic tumor area delineation and tumor tissue characterization are realized, thereby greatly reducing the dependence on the experience of the operating personnel and the sonographers, lightening the workload of the sonographers, and improving the accuracy and efficiency of tumor area delineation. Thus, a new method is provided for tumor tissue characterization. This method has the advantages of automatic fast scanning, automatic tumor delineation, automatic tumor tissue analysis, saving of medical resources, etc.

### Embodiment 2

A computer device includes a memory, a processor, and a computer program stored on the memory and runnable on the processor, where the processor is configured to execute the computer program to implement the breast tissue characterization method in Embodiment 1.

### Embodiment 3

A computer-readable storage medium stores thereon a computer program which, when executed by a processor, implements the breast tissue characterization method in Embodiment 1.

### Embodiment 4

A computer program product includes a computer program which, when executed by a processor, implements the breast tissue characterization method in Embodiment 1.

### Embodiment 5

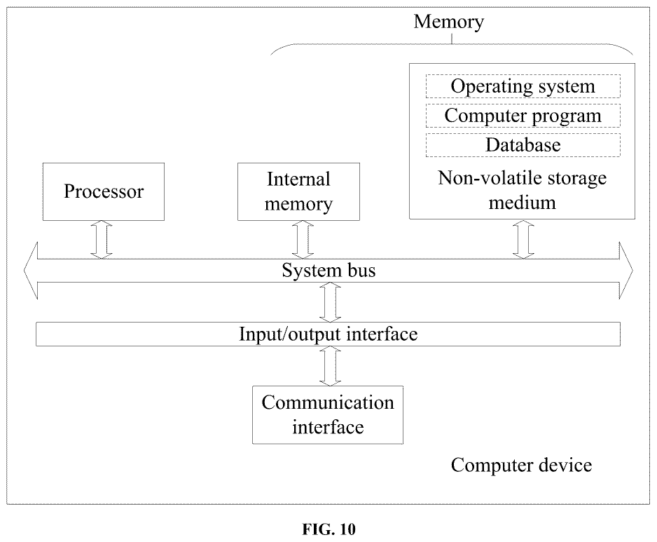

A computer device is provided, which may have an internal structure as shown in FIG. 10. The computer device includes a processor, a memory, an input/output interface (I/O), and a communication interface. The processor, the memory, and the input/output interface are connected through a system bus, and the communication interface is connected to the system bus through the input/output interface. The processor of the computer device is configured to provide computing and control capabilities. The memory of the computer device includes a nonvolatile storage medium and an internal memory. The non-volatile storage medium stores an operating system, a computer program, and a database. The internal memory provides an environment for operation of the operating system and the computer program in the nonvolatile storage medium. The database of the computer device is configured to store pending transactions. The input/output interface of the computer device is configured to exchange information between the processor and an external device. The communication interface of the computer device is configured to communicate with an external terminal through a network. The computer program, when executed by a processor, is configured to, implement the breast tissue characterization method in Embodiment 1.

Those of ordinary skill in the art may understand that all or some of the procedures in the methods of the above embodiments may be implemented by a computer program instructing related hardware. The computer program may be stored in a nonvolatile computer-readable storage medium. When the computer program is executed, the procedures in the embodiments of the above methods may be performed. Any reference to the memory, the database, or other media used in the embodiments of the present disclosure may include at least one of a nonvolatile memory and a volatile memory. The nonvolatile memory may include a read-only memory (ROM), a magnetic tape, a floppy disk, a flash memory, an optical memory, a high-density embedded nonvolatile memory, a resistive random access memory (ReRAM), a magnetoresistive random access memory (MRAM), a ferroelectric random access memory (FRAM), a phase change memory (PCM), a graphene memory, etc. The volatile memory may include a random access memory (RAM) or an external cache memory. As an illustration rather than a limitation, the RAM may be in various forms, such as a static random access memory (SRAM) or a dynamic random access memory (DRAM). The database in the embodiments of the present disclosure may include at least one of a relational database and a non-relational database. The non-relational database may include a distributed database based on a blockchain, but is not limited thereto. The processor involved in the embodiments of the present disclosure may be a general-purpose processor, a central processing unit, a graphics processing unit, a digital signal processor, a programmable logic device, or the like, and is not limited thereto.

The technical features of the foregoing embodiments can be combined arbitrarily. For brevity of description, not all possible combinations of the technical features of the foregoing embodiments are described. However, the combinations of these technical features should be construed as falling within the scope described in this specification as long as there is no contradiction between the combinations.

Particular examples are used herein for illustration of principles and implementation modes of the present disclosure. The descriptions of the above embodiments are merely used for assisting in understanding the method of the present disclosure and its core ideas. In addition, those of ordinary skill in the art can make various modifications in terms of particular implementation modes and the scope of application in accordance with the ideas of the present disclosure. In conclusion, the content of the description shall not be construed as limitations to the present disclosure.

What is claimed is:

1. Abreast tissue characterization method, comprising:
acquiring three-dimensional data of a breast;
determining signals of sections of the breast based on the three-dimensional data of the breast, wherein the sections comprise a cross section, a sagittal section, and a coronal section, and the signals comprise a radio frequency (RF) signal, an envelope signal, and a B-mode signal;
generating a corresponding B-mode image based on the determined B-mode signal of each section;
inputting the B-mode images of the sections to a region of interest (ROI) detection model to obtain a tumor ROI area, wherein the ROI detection model comprises a tumor B-mode edge detection model configured to input the B-mode images of the sections and output a tumor B-mode edge, a tracking module configured to obtain a tracked tumor envelope edge by tracking based on the tumor B-mode edge and obtain a tracked tumor RF edge by tracking based on the tracked tumor envelope edge, and an ROI generation module configured to determine the tumor ROI area based on the tumor B-mode edge, the tracked tumor envelope edge, and the tracked tumor RF edge;
extracting target signals from the three-dimensional data of the breast, wherein the target signals comprise RF signals, envelope signals, and B-mode signals respectively corresponding to a cross section ROI, a sagittal section ROI, and a coronal section ROI that are determined based on the tumor ROI area; the cross section ROI comprises a region of a tumor in the cross section, the sagittal section ROI comprises a region of the tumor in the sagittal section, and the coronal section ROI comprises a region of the tumor in the coronal section; and
inputting the target signals to a tumor tissue analysis model and outputting a final tumor tissue characterization result by the tumor tissue analysis model, wherein the final tumor tissue characterization result comprises a tumor morphology, a tumor aspect ratio, a tumor edge, tumor internal echo and tumor posterior echo characteristics, a calcification characteristic for characterizing whether the tumor is internally calcified, an invasiveness characteristic for characterizing whether the tumor is invasive, and a metastasis characteristic for characterizing whether the tumor metastasizes;
wherein the tumor tissue analysis model comprises a quantitative characterization module configured to extract specific features of the tumor based on the target signals, a trained neural network configured to obtain a preliminary tumor tissue characterization result based on the target signals, and a trained systematic learning network configured to output the final tumor tissue characterization result based on the specific features and the preliminary tumor tissue characterization result.

2. The breast tissue characterization method according to claim 1, wherein the ROI detection model is determined according to the following steps:
acquiring first training data and a first label, wherein the first training data comprises the B-mode images of the sections of the breast determined based on the three-dimensional data of the breast as a sample, and the first label comprises a true tumor B-mode edge;
constructing a target model, wherein the target model comprises a first neural network for tumor edge detection, the tracking module, a tracing module, and the ROI generation module; an output of the first neural network for tumor edge detection is connected to an input to the tracking module; outputs of the first neural network for tumor edge detection, the tracking module, and the tracing module are all connected to the ROI generation module; the tracing module is configured to obtain a corresponding traced tumor envelope edge by tracing based on an input predicted tumor RF edge and obtain a traced tumor B-mode edge by tracing based on the obtained traced tumor envelope edge, and the predicted tumor RF edge is determined by a tumor RF edge detection model based on RF images corresponding to the B-mode images in the training data;
training the first neural network for tumor edge detection with the first training data and the first label for a plurality of times with an objective of minimizing a total loss to obtain the tumor B-mode edge detection model;
wherein a total loss in any training process is determined based on an error value that comprises: an error between the tumor B-mode edge output by the first neural network for tumor edge detection obtained by last training and the true tumor B-mode edge, an error between the tracked tumor envelope edge generated by the tracking module and the predicted tumor envelope edge, an error between the tracked tumor RF edge generated by the tracing module and the predicted tumor RF edge, an error between the traced tumor envelope edge generated by the tracing module and the predicted tumor envelope edge, and an error between the traced tumor B-mode edge generated by the tracing module and the true tumor B-mode edge; and
connecting an output of the tumor B-mode edge detection model to the input to the tracking module, and connecting the outputs of the tumor B-mode edge detection model and the tracking module to the ROI generation module to obtain the ROI detection model.

3. The breast tissue characterization method according to claim 2, wherein the error value further comprises an error between the tumor B-mode edge output by the first neural network for tumor edge detection obtained by last training and an artificial feedback tumor edge determined based on artificial feedback information for the tumor ROI area.

4. The breast tissue characterization method according to claim 1, wherein the quantitative characterization module comprises a first quantitative characterization module, a second quantitative characterization module, and a third quantitative characterization module, wherein:
the first quantitative characterization module is configured to extract a first specific feature of the tumor based on the RF signals of the target signals;
the second quantitative characterization module is configured to extract a second specific feature of the tumor based on the envelope signals of the target signals; and
the third quantitative characterization module is configured to extract a third specific feature of the tumor based on the B-mode signals of the target signals.

5. The breast tissue characterization method according to claim 1, wherein the trained neural network comprises a first trained neural network, a second trained neural network, and a third trained neural network, wherein:
the first trained neural network is configured to obtain, based on the RF signals of the target signals, a preliminary tumor tissue characterization result corresponding to the RF signals;
the second trained neural network is configured to obtain, based on the envelope signals of the target signals, a preliminary tumor tissue characterization result corresponding to the envelope signals; and
the third trained neural network is configured to obtain, based on the B-mode signals of the target signals, a preliminary tumor tissue characterization result corresponding to the B-mode signals.

6. The breast tissue characterization method according to claim 1, wherein the trained systematic learning network specifically comprises a feature selection module, a trained first-layer primary learner, and a trained second-layer meta learner that are connected in sequence;
the feature selection module is configured to select, using a mutual information method, the specific features and preliminary tumor tissue characterization results to obtain a selection result;
the trained first-layer primary learner is configured to input the selection result and output a primary tumor tissue characterization result; and
the trained second-layer meta learner is configured to input the primary tumor tissue characterization result and output the final tumor tissue characterization result.

7. The breast tissue characterization method according to claim 1, wherein the determining signals of sections of the breast specifically comprises:
extracting the RF signal of each section of the breast from the three-dimensional data of the breast;
performing pre-gain amplification on the RF signal of each section, demodulating the amplified signal using an in-phase and quadrature demodulation algorithm, and detecting the demodulated signal by hilbert transform to obtain the envelope signal of each section of the breast; and
performing logarithmic compression on the envelope signal of each section to obtain the B-mode signal of each section of the breast.

8. The breast tissue characterization method according to claim 1, wherein the acquiring three-dimensional data of a breast specifically comprises:
scanning the breast using a multimodal breast volumetric ultrasound integrated machine to obtain the three-dimensional data of the breast.

9. The breast tissue characterization method according to claim 1, wherein the extracting target signals from the three-dimensional data of the breast specifically comprises:
receiving artificial calibration information for the tumor ROI area;
determining a final tumor ROI area based on the artificial calibration information, wherein the final tumor ROI area comprises the cross section ROI, the sagittal section ROI, and the coronal section ROI; and
extracting from the three-dimensional data of the breast the RF signal, the envelope signal, and the B-mode signal corresponding to the cross section ROI, the RF signal, the envelope signal, and the B-mode signal corresponding to the sagittal section ROI, and the RF signal, the envelope signal, and the B-mode signal corresponding to the coronal section ROI.

10. The breast tissue characterization method according to claim 4, wherein:
the first specific feature comprises a complexity related feature, a texture related feature, a frequency domain related feature, and a blood flow doppler related feature;
the second specific feature comprises a shape parameter and a scale parameter in a distribution model; and
the third specific feature comprises an elasticity imaging related parameter, a texture related feature, a morphology related feature, and an image gray level related feature.

11. A computer device, comprising a memory, a processor, and a computer program stored on the memory and runnable on the processor, wherein the processor is configured to execute the computer program to implement the breast tissue characterization method according to claim 1.

12. A non-transitory computer-readable storage medium, storing thereon a computer program that, when executed by a processor, implements the breast tissue characterization method according to claim 1.

13. The computer device according to claim 11, wherein the ROI detection model is determined according to the following steps:
acquiring first training data and a first label, wherein the first training data comprises the B-mode images of the sections of the breast determined based on the three-dimensional data of the breast as a sample, and the first label comprises a true tumor B-mode edge;
constructing a target model, wherein the target model comprises a first neural network for tumor edge detection, the tracking module, a tracing module, and the ROI generation module; an output of the first neural network for tumor edge detection is connected to an input to the tracking module; outputs of the first neural network for tumor edge detection, the tracking module, and the tracing module are all connected to the ROI generation module; the tracing module is configured to obtain a corresponding traced tumor envelope edge by tracing based on an input predicted tumor RF edge and obtain a traced tumor B-mode edge by tracing based on the obtained traced tumor envelope edge, and the predicted tumor RF edge is determined by a tumor RF edge detection model based on RF images corresponding to the B-mode images in the training data;
training the first neural network for tumor edge detection with the first training data and the first label for a plurality of times with an objective of minimizing a total loss to obtain the tumor B-mode edge detection model;
wherein a total loss in any training process is determined based on an error value that comprises: an error between the tumor B-mode edge output by the first neural network for tumor edge detection obtained by last training and the true tumor B-mode edge, an error between the tracked tumor envelope edge generated by the tracking module and the predicted tumor envelope edge, an error between the tracked tumor RF edge generated by the tracing module and the predicted tumor RF edge, an error between the traced tumor envelope edge generated by the tracing module and the predicted tumor envelope edge, and an error between the traced tumor B-mode edge generated by the tracing module and the true tumor B-mode edge; and
connecting an output of the tumor B-mode edge detection model to the input to the tracking module, and connecting the outputs of the tumor B-mode edge detection model and the tracking module to the ROI generation module to obtain the ROI detection model.

14. The computer device according to claim 13, wherein the error value further comprises an error between the tumor B-mode edge output by the first neural network for tumor edge detection obtained by last training and an artificial feedback tumor edge determined based on artificial feedback information for the tumor ROI area.

15. The computer device according to claim 11, wherein the quantitative characterization module comprises a first quantitative characterization module, a second quantitative characterization module, and a third quantitative characterization module, wherein:
the first quantitative characterization module is configured to extract a first specific feature of the tumor based on the RF signals of the target signals;
the second quantitative characterization module is configured to extract a second specific feature of the tumor based on the envelope signals of the target signals; and
the third quantitative characterization module is configured to extract a third specific feature of the tumor based on the B-mode signals of the target signals.

16. The computer device according to claim 11, wherein the trained neural network comprises a first trained neural network, a second trained neural network, and a third trained neural network, wherein:
the first trained neural network is configured to obtain, based on the RF signals of the target signals, a preliminary tumor tissue characterization result corresponding to the RF signals;
the second trained neural network is configured to obtain, based on the envelope signals of the target signals, a preliminary tumor tissue characterization result corresponding to the envelope signals; and
the third trained neural network is configured to obtain, based on the B-mode signals of the target signals, a preliminary tumor tissue characterization result corresponding to the B-mode signals.

17. The computer device according to claim 11, wherein the trained systematic learning network specifically comprises a feature selection module, a trained first-layer primary learner, and a trained second-layer meta learner that are connected in sequence;
the feature selection module is configured to select, using a mutual information method, the specific features and preliminary tumor tissue characterization results to obtain a selection result;
the trained first-layer primary learner is configured to input the selection result and output a primary tumor tissue characterization result; and
the trained second-layer meta learner is configured to input the primary tumor tissue characterization result and output the final tumor tissue characterization result.

18. The computer device according to claim 11, wherein the determining signals of sections of the breast specifically comprises:
extracting the RF signal of each section of the breast from the three-dimensional data of the breast;
performing pre-gain amplification on the RF signal of each section, demodulating the amplified signal using an in-phase and quadrature demodulation algorithm, and detecting the demodulated signal by hilbert transform to obtain the envelope signal of each section of the breast; and
performing logarithmic compression on the envelope signal of each section to obtain the B-mode signal of each section of the breast.

19. The computer device according to claim 11, wherein the acquiring three-dimensional data of a breast specifically comprises:
scanning the breast using a multimodal breast volumetric ultrasound integrated machine to obtain the three-dimensional data of the breast.

20. The computer device according to claim 11, wherein the extracting target signals from the three-dimensional data of the breast specifically comprises:
receiving artificial calibration information for the tumor ROI area;
determining a final tumor ROI area based on the artificial calibration information, wherein the final tumor ROI area comprises the cross section ROI, the sagittal section ROI, and the coronal section ROI; and
extracting from the three-dimensional data of the breast the RF signal, the envelope signal, and the B-mode signal corresponding to the cross section ROI, the RF signal, the envelope signal, and the B-mode signal corresponding to the sagittal section ROI, and the RF signal, the envelope signal, and the B-mode signal corresponding to the coronal section ROI.
